(19) United States
(12) Patent Application Publication
(10) Pub. No.: US 2025/0381362 A1
(43) Pub. Date: Dec. 18, 2025

(54) VIRTUAL ENVIRONMENT SMELL SIMULATION SYSTEM

(71) Applicant: Mesut Yilmaz, Herne (DE)

(72) Inventor: Mesut Yilmaz, Herne (DE)

(21) Appl. No.: 19/032,076

(22) Filed: Jan. 19, 2025

Related U.S. Application Data
(60) Provisional application No. 63/623,029, filed on Jan. 19, 2024.

(51) Int. Cl. A61M 21/00; A61N 1/36; G06F 3/01; G06Q 30/0251

(52) U.S. Cl. CPC A61M 21/00; A61N 1/36014; G06F 3/011; G06Q 30/0261; A61M 2021/0016; A61M 2021/0072; A61M 2209/088

(57) ABSTRACT

A smell simulation system includes an olfactory stimulation system comprising an olfactory stimulation delivery device containing an olfactory stimulator. The olfactory stimulation delivery device is adapted to position the olfactory stimulator on or in proximity to the user so that the olfactory stimulator can provide an electrical stimulus to the user that generates an olfactory effect. A connection system is adapted to connect the olfactory stimulation system to a virtual environment, wherein the olfactory stimulation system provides an olfactory stimulation to the user in response to an interaction with the virtual environment.

### PRIORITY

The present application claims the benefit of domestic priority based on U.S. Provisional Patent Application 63/623,029 filed on Jan. 19, 2024, the entirety of which is incorporated herein by reference.

### BACKGROUND

When a user interacts with or within a virtual environment, it is desirable for the user's experience to be as lifelike as possible.

Virtual environments of all sorts currently exist where a user is immersed within a setting other than or in addition to the user's physical environment. A virtual environment is an environment or partial environment generated electronically, such as by computer graphics or other sensory stimuli and presented to the user by a delivery system, such as a display screen, speaker system, holographic system, and the like.

For decades designers of virtual environment systems have been striving to make the user's experience of the virtual environment as realistic as possible. One way to increase realism is through sensory feedback. Visual, auditory, and/or tactile feedback systems have been developed that allow a user to interact within a virtual environment and experience it through one or more of the user's senses. However, heretofore the ability for a virtual environment system to interact with a user's olfactory system and the user's sense of smell has been less than ideal. Accordingly, a user virtually walking through a field of flowers or viewing a chef cook a meal on a cooking website has been unable to effectively experience the smells of the virtual environment. The user can see the beautiful flowers or see and hear bacon sizzling in a pan, but the simulated experience is not complete without the odors one would expect to accompany the sights or sounds that are being presented. Without such olfactory sensation, the experience is not as real as it could be.

Previous attempts at providing olfactory simulation in a virtual environment have fallen short and have failed to catch on. For example, U.S. Pat. No. 9,925,549 discloses a head-mounted scent delivery system that is configured to dispense scented materials in response to a scene that is shown to a user. The scented materials are held in capsules and are dispensed based on the characteristics of the content being provided to the user. However, this system is cumbersome to wear, and its effectiveness is limited by the variety of scents that are available in the capsules. In addition, the system is not instantaneously responsive to the virtual environment in that there can be a delay between when a material is instructed to be dispensed and when a user actually smells the material. Also, there can be a lingering effect where a scent lingers longer than it should based on a change in the virtual environment.

There is therefore a need for an improved system for experiencing a virtual environment where a more realistic experience can be provided to a user. There is further a need for an improved system for experiencing a virtual environment in which a smell can be simulated for a user. There is a further need for a system that can provide improved olfactory stimulation to a user in a virtual environment where the olfactory stimulation or simulation is responsive to the virtual environment. There is a further need for providing olfactory stimulation or simulation to a user in a virtual environment in a convenient, comfortable, non-invasive, and/or responsive manner.

### SUMMARY

The present invention satisfies these needs. In one aspect of the invention, an improved system for experiencing a virtual environment is provided.

In another aspect of the invention, a system for experiencing a virtual environment provides an improvement in the realistic experience provided to a user.

In another aspect of the invention, a system for interacting with a virtual environment provides an improvement in the realistic experience provided to a user.

In another aspect of the invention, a system provides an improved olfactory simulation or perception to a user in a virtual environment.

In another aspect of the invention, a system provides an improved olfactory simulation or perception to a user in a virtual environment where the olfactory stimulation is responsive to the virtual environment.

In another aspect of the invention, a system provides olfactory stimulation and/or simulation to a user in a virtual environment in a convenient, comfortable, and/or responsive manner.

In another aspect of the invention, a smell simulation system comprises an olfactory stimulation system and a connection system for electronically connecting to a virtual environment.

In another aspect of the invention, a smell simulation system comprises an olfactory stimulation system and a connection system for electronically connecting to a virtual environment so that the olfactory stimulation system can be responsive to and/or interact with the virtual environment.

In another aspect of the invention, a smell simulation system comprises an olfactory stimulation system comprising an olfactory stimulation delivery device that is adapted to be worn by or positionable near a user that is engaging with or within the virtual environment, the olfactory stimulation delivery device having one or more olfactory stimulators mounted or positionable thereon in a location where the one or more olfactory stimulators can deliver a stimulus to the user in a manner that stimulates an olfactory response in the user to cause the user to experience a simulated smell and/or a perceived smell sensation.

In another aspect of the invention, a smell simulation system comprises an olfactory stimulation system comprising an olfactory stimulation delivery device that is adapted to be worn by or positionable near a user that is engaging with or within the virtual environment, the olfactory stimulation delivery device having one or more olfactory stimulators mounted or positionable thereon in a location where the one or more olfactory stimulators can deliver a stimulus to the user in a manner that stimulates an olfactory response in the user to cause the user to experience a simulated smell and/or a perceived smell sensation, the olfactory stimulation device comprising a head set and one or more electrodes.

In another aspect of the invention, a smell simulation system comprises an olfactory stimulation system comprising an olfactory stimulation delivery device that is adapted to be worn by or positionable near a user that is engaging with or within the virtual environment, the olfactory stimulation delivery device having one or more olfactory stimulators mounted or positionable thereon in a location where the one or more olfactory stimulators can deliver a stimulus to the user in a manner that stimulates an olfactory response in the user to cause the user to experience a simulated smell and/or a perceived smell sensation, the olfactory stimulation device comprising a head set and one or more electrodes in the form of one or more non-invasive electrical stimulus emitters that are positionable to deliver an electrical stimulus to, near, or through an external surface of the user.

In another aspect of the invention, a smell simulation system comprises an olfactory stimulation system comprising an olfactory stimulation delivery device that is adapted to be worn by or positionable near a user that is engaging with or within the virtual environment, the olfactory stimulation delivery device having one or more olfactory stimulators mounted or positionable thereon in a location where the one or more olfactory stimulators can deliver a stimulus to the user in a manner that stimulates an olfactory response in the user to cause the user to experience a simulated smell and/or a perceived smell sensation, the olfactory stimulation device comprising a head set and one or more electrodes that are adapted to deliver electromagnetic pulses to the user, the electromagnetic pulses being deliverable to the olfactory bulb of the user to cause the brain of the user to perceive the detection of a particular smell.

In another aspect of the invention, a smell simulation system comprises an olfactory stimulation system comprising an olfactory stimulation delivery device that is adapted to be worn by or positionable near a user that is engaging with or within the virtual environment, the olfactory stimulation delivery device having one or more olfactory stimulators mounted or positionable thereon in a location where the one or more olfactory stimulators can deliver a stimulus to the user in a manner that stimulates an olfactory response in the user to cause the user to experience a simulated smell and/or a perceived smell sensation, the olfactory stimulation device comprising a head set and one or more electrodes, the one or more electrodes being positionable at one or more positions, such as one or more of either side of the bridge of the nose, the posterior side of the scalp, the center of the lower forehead, the neck posterior.

In another aspect of the invention, a smell simulation system comprises an olfactory stimulation system that provides an olfactory simulation or perception to a user viewing a webpage on the internet.

In another aspect of the invention, a smell simulation system comprises an olfactory stimulation system that provides an olfactory simulation or perception to a user viewing a webpage on the internet, the olfactory simulation or perception being in response to an image or signal detected on or coming from the webpage.

In another aspect of the invention, a smell simulation system comprises an olfactory stimulation system that provides an olfactory simulation or perception to a user interacting with an application.

In another aspect of the invention, a smell simulation system comprises an olfactory stimulation system that provides an olfactory simulation or perception to a user interacting with an application, the olfactory simulation or perception being in response to an image or signal detected on or coming from the application.

In another aspect of the invention, a smell simulation system comprises an olfactory stimulation system that provides an olfactory simulation or perception to a user interacting with an electronic game.

In another aspect of the invention, a smell simulation system comprises an olfactory stimulation system that provides an olfactory simulation or perception to a user interacting with an electronic game, the olfactory simulation or perception being in response to an image or signal detected on or coming from the electronic game.

In another aspect of the invention, a method of simulating a smell to a user of a virtual environment comprises providing a smell simulation system as disclose above and/or below and using the smell simulation system in any manner described herein.

In another aspect of the invention, a smell simulation system comprises an olfactory stimulation system comprising an olfactory stimulation delivery device containing an olfactory stimulator, wherein the olfactory stimulation delivery device is adapted to position the olfactory stimulator on or in proximity to the user so that the olfactory stimulator can provide an electrical stimulus to the user that generates an olfactory effect; and a connection system adapted to connect the olfactory stimulation system to a virtual environment, wherein the olfactory stimulation system provides an olfactory stimulation to the user in response to an interaction with the virtual environment.

In another aspect of the invention, a smell simulation process comprises providing a virtual environment for a user; and providing an electrical stimulation to the user in response to the user's interaction within the virtual environment, wherein the electrical stimulation is designed to produce a simulated olfactory effect related to the virtual interaction.

In another aspect of the invention, a smell simulation process comprises providing a virtual environment for a user; and providing olfactory stimulation to the user in response to the user's interaction with an advertisement within the virtual environment, wherein the olfactory stimulation is designed to produce a simulated olfactory effect related to the advertisement.

In another aspect of the invention, a smell simulation process comprises providing a virtual environment for a user; and providing olfactory stimulation to the user in response to the user's interaction with an advertisement within the virtual environment, wherein the olfactory stimulation is designed to produce a simulated olfactory effect related to the advertisement, and wherein the olfactory stimulation is logged and monetized.

### BRIEF DESCRIPTION OF THE DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIG. 1 is a schematic diagram of a smell simulation system of the invention;

FIG. 2 is a schematic diagram of a virtual environment system incorporating a smell simulation system of the invention;

FIG. 3 is a schematic flow chart of an olfactory feedback process using a smell simulation system of the invention;

FIG. 4 is a schematic front view of a version of an olfactory stimulation system that may be used with a smell simulation system of the invention;

FIG. 5 is a schematic side view of another version of an olfactory stimulation system that may be used with a smell simulation system of the invention;

FIG. 6 is a schematic flow chart of another version of an olfactory feedback process using a smell simulation system of the invention;

FIG. 7 is a schematic flow chart of another version of an olfactory feedback process using a smell simulation system of the invention;

FIG. 8 is a schematic flow chart of another version of an olfactory feedback process using a smell simulation system of the invention; and

FIG. 9 is a schematic flow chart of another version of an olfactory feedback process using a smell simulation system of the invention.

### DESCRIPTION

The present invention relates to providing smell simulation for a user in a virtual environment. In particular, the invention relates to a system and method for providing olfactory stimulation to a user in a virtual environment. Although the invention is illustrated and described in the context of being useful for interactive virtual environment systems, such as a virtual reality system and/or a website, the present invention can be useful in other instances. Accordingly, the present invention is not intended to be limited to the examples and embodiments described herein.

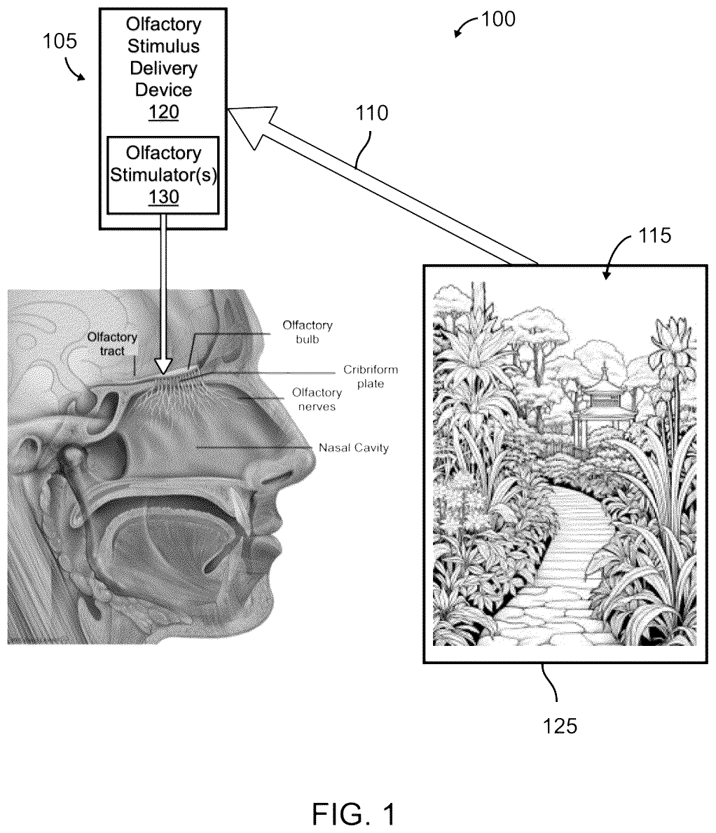

FIG. 1 shows a smell simulation system 100 of the invention. The smell simulation system 100 includes an olfactory stimulation system 105 and a connection system 110 for electronically connecting to a virtual environment 115 so that the olfactory stimulation system 105 can be responsive to and/or interact with the virtual environment 115. The olfactory stimulation system 105 includes an olfactory stimulation delivery device 120 that is adapted to be worn by or positionable near a user that is engaging with or within the virtual environment 115, such as a virtual environment presented to a user by a display 125. The olfactory stimulation delivery device 120 has one or more olfactory stimulators 130 mounted or positionable thereon in a location where the one or more olfactory stimulators 130 can deliver a stimulus, such as an electrical stimulus, to the user in a manner that stimulates an olfactory response in the user to cause the user to experience a simulated olfactory effect, such as a simulated smell and/or a perceived smell sensation. In one version, the olfactory stimulation system 105 provides a stimulus that is delivered to the olfactory bulb or region of a user's brain in a manner that causes the user to perceive a smell sensation. By virtual environment it is meant any computer or electronically generated sensory display or presentation that is provided to a user that is different than and/or an enhancement of a user's physical environment. Virtual environments can range from very complex systems, such as movable cockpit flight simulators, to more simple systems, such as a handheld gaming device or audio book. Other examples of virtual environments include extended reality systems, virtual reality systems, augmented reality systems, mixed reality systems, websites, gaming devices, televisions, radios, smart home applications, and the like. Commonly, and as exemplified hereinbelow, a virtual environment 115 can be a computerized virtual reality application, such as a game or reality simulation, that is presented to the user on or through a display 125, such as a display screen, such as a computer monitor, smart phone device, and/or a head mounted virtual reality display device.

The smell simulation system 100 presents to the user an interaction in the virtual environment 115 with improved realness. Immersive displays, such as a head mounted virtual reality displays, can provide an incredible amount of realism in virtual environments, such as gaming applications, by stimulating and engaging the visual and/or auditory senses. With the smell simulation system 100, the realism can be taken to an even higher level by being able to stimulate the olfactory region and thereby simulate smells that a user can experience while in the virtual environment 105. With the olfactory stimulation system 105 of the smell simulation system 100 it is possible to add a perceptible olfactory experience for the user by providing simulated olfactory effects or cues that have a relationship with the user's virtual interactions in the virtual environment 110. Thus, whereas typical sensory feedback systems provide a visual, auditory, and/or tactile sensation for the user, olfactory cues can provide smell sensation feedback to go along with one or more of those sensations.

The olfactory stimulation system 105 can generate and provide an olfactory stimulation to a user though the one or more olfactory stimulators 130. For example, the olfactory stimulation system 105 can provide an electrical stimulation of the olfactory region of a user to generate an olfactory sensation for a user while the user is interacting with the virtual environment 115. In one version, the olfactory stimulation system 105 can generate an electrical stimulation that is in response to an interaction within the virtual environment 115. In one version, the olfactory stimulation system 105 can generate a first electrical stimulation that is in response to a first interaction within the virtual environment 115 and can generate a second electrical stimulation that is different than the first electrical stimulation and that is in response to a second interaction within the virtual environment 115. In this version, the first electrical stimulation can be designed to simulate a first olfactory effect, such as a first odor or smell sensation, and the second electrical stimulation can be designed to simulate a second olfactory effect, such as a second odor or smell sensation. Accordingly, a modulated smell sensation can be provided to a user based on and in response to a user's interaction with a virtual environment 115.

In one version, as mentioned above, the olfactory stimulation system 105 can interact with a virtual environment application, such as a computer game, an internet site, or the like. One or more sensors can determine a user's interaction with the application in known manner and an olfactory feedback signal can be generated that is related to an olfactory occurrence in the virtual environment. The olfactory feedback signal causes the olfactory stimulation system 105 to provide olfactory feedback to the user through the one or more olfactory stimulators 130. This creates a more immersive and real experience for the user since the virtual environment 115 can be sensed through a simulated smell sensation in addition to the conventional sensory inputs that are available.

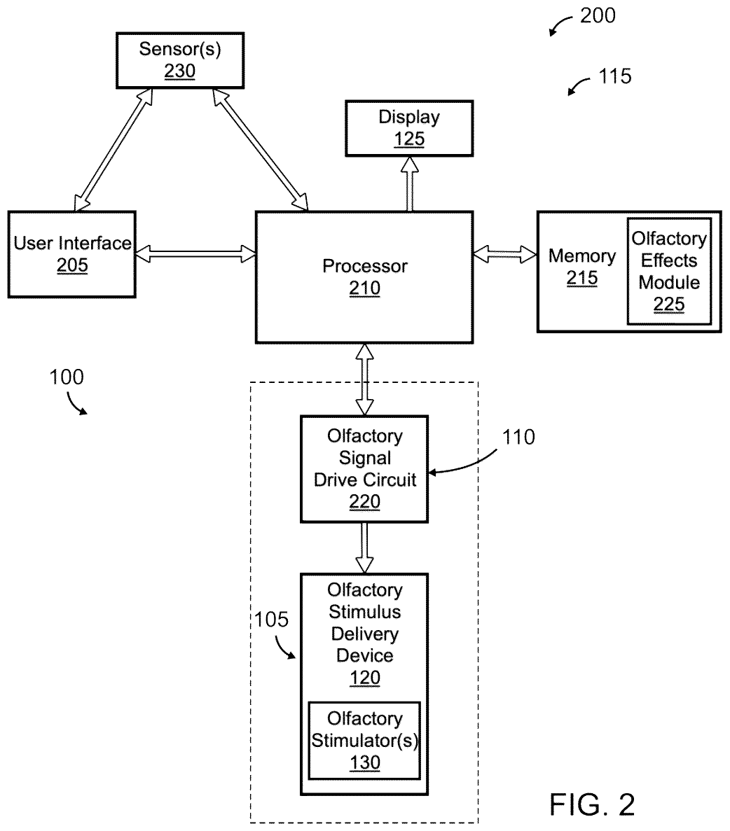

FIG. 2 shows a conceptual block diagram of a virtual environment system 200 that incorporates a smell simulation system 100 of the invention. The virtual environment system 200 may be an interactive virtual environment system that includes a user interface 205, which may be in the form of a button, keys, keyboard, game controller, movement sensors, and the like. The virtual environment system 200 of the version of FIG. 2 includes a processor or controller 210 that is coupled to a memory 215 and to an olfactory signal drive circuit 220 which is in communication with the olfactory stimulation system 105. The processor or controller 210 may be any type of general purpose processor or could be a processor specifically designed to provide smell sensation feedback effects, such as an application-specific integrated circuit (“ASIC”). The processor or controller 210 may be the same processor that operates the entire virtual environment system 200 or may be a separate processor. The processor or controller 210 can decide what olfactory simulated effects are to be provided to the user and the order in which the effects are provided based on programmed parameters. For example, the programmed parameters can include the type of olfactory effect to be provided, the magnitude of the olfactory effect, and/or the duration of the olfactory effect. The olfactory effect may be considered to be a dynamic olfactory effect if it includes some variation of these parameters when the olfactory effect is generated or a variation of these parameters based on a user's interaction.

The processor or controller 210 outputs a control signal to olfactory signal drive circuit 220 which includes electronic components and circuitry used to supply the olfactory stimulation system 105 with the required electrical current and voltage, i.e. olfactory signals, to cause a desired olfactory effect to be present to the user by the olfactory stimulation system 105 to generate an olfactory simulated effect. The memory 215 can be any type of storage device or computer-readable medium, such as random access memory (“RAM”) or read-only memory (“ROM”). The memory 215 can store instructions executed by the processor or controller 210. For example, the memory 215 can include an olfactory effects module 225 which includes instructions that, when executed by processor 210, generate drive signals for the olfactory signal drive circuit 220 to provide to the olfactory stimulation system 105 so that it can provide olfactory effects to the user, as disclosed in more detail below. Memory 215 may also be located internal to processor or controller 210 and/or may be housed within the smell simulation system 100. The olfactory signal drive circuit 220 may communicate with the olfactory stimulation system 105 in any known manner, such as my electrical wiring or wireless communication.

The user interface 205 recognizes user interactions, such as touches with a device or manipulations of virtual objects in a virtual reality application. A manipulation of a virtual object can include any perceived contact with a virtual object using virtual hands (or other virtual implements) available to and controlled by a user in a virtual environment 115. In other applications, manipulation can include control of an element of the application by a user using a user interface. The application would typically provide visual and/or auditory feedback to the user that tracks the user interaction and guides the user with its feedback. The user interface 205 can also allow the user to move within the virtual environment 115 and change position within the virtual environment 115 and/or change the environment setting. In this regard, the user interface 205 can recognize and control all sorts of interactions between the user and the virtual environment 115. For example, the user interface 205 can be manipulated by the user to cause a simulation of picking up and looking at an apple and then picking up and looking at an orange. The virtual environment 200 can allow the user to experience the sights and/or touch sensations of these maneuvers, in conventional manner, and with the incorporation of the smell simulation system 100 can also allow the user to experience the olfactory effects, or olfactory simulated effects, such as smells of the virtual apple and/or orange. In one version, the olfactory signal and thus the olfactory stimulation that is provided to the user can be modulated in accordance with the interaction. For example, when the user virtually picks up the apple, the simulated smell can be caused to grow more intense as the apple is brough closer to the virtual nose in the simulation.

The virtual environment system 200 may also include one or more sensors 230 that can operate independent of or in conjunction with the user interface 205 and that are adapted to sense interactions between the user and the virtual environment 115. The sensors 230 may be present within a handheld controller or phone or otherwise positioned on the user. The sensors 230 may, for example, be degrees of freedom sensors detecting up to the six degrees of motion including one or more of up/down, back/forward, right/left, roll, pitch, and yaw. Such sensors 230 can include magnetic sensors, electromagnetic field sensors, accelerometers, gyroscopes, and others for detecting positional and angular data. Force sensing resistor sensors and multi-touch pressure sensors can measure the pressure applied under each touch location. Other types of sensors 230 may additionally or alternatively be used, such as temperature, humidity, and atmospheric pressure sensors that can capture environmental conditions or a microphone that can capture a user's voice command or environmental audio information. The data corresponding to the one or more sensors 230 is sent to processor or controller 210, or another processor within the virtual environment system 200, and the processor or controller 210 interprets the sensor data and in response can generate olfactory signals or recall olfactory signals from the olfactory effects module 225.

The virtual environment system 200 may be, for example, a desktop or laptop computer, a handheld device, such a cellular telephone, personal digital assistant (“PDA”), smartphone, computer tablet, gaming console, vehicle based interface, or the like. The virtual environment 200 may be used with a virtual reality geer, including a display device and one or more sensors that track movement of a user and/or movement of a user's hands. User interface 205 may be a touch sensitive surface, or can be any other type of user interface such as a keyboard, mouse, touchpad, mini-joystick, scroll wheel, trackball, game pads or game controllers, gloves with integrated or mounted sensors, motion tracking cameras, etc. As discussed above, the virtual environment can be an application, a game, an internet website or webpage, or any other virtual environment.

In addition to or alternatively to providing user interfacing olfactory effects in response to user interaction, the virtual environment system 200 may provide statically generated olfactory effects for playback along with, for example, a video or audio file. One example of such a system includes a device that plays a video or series of photographs to a user via a display 125, such as a display screen, and that is connected to an olfactory stimulation system 105 that is in communication with the user. At a predetermined time in the video, the olfactory stimulation system 105 can be caused to stimulate the olfactory system of the user to generate an olfactory simulated effect, such as the perception of a smell. By coordinating the timing of the stimulation and the video playback, the user can simultaneously receive olfactory and visual simulation sensations and thus an enhanced virtual experience.

The smell simulation system 100 can also be used to add olfactory stimulation to a system that lacks any olfactory stimulation. Such olfactory cues can be more beneficial than not having any olfactory cues and can be used to enhance or alter the virtual environment experience. Some examples of immersive displays include the “Oculus Rift” by Oculus VR, Inc. and the “Head Mounted Display” or “Wearable HDTV” by Sony Electronics Inc., the “Project Morpheus” head mounted display, by Sony Computer Entertainment Inc., and the “Vision Pro” by Apple. Olfactory cues add additional realistic elements to interactions that use these immersive displays. Users can interact with an application in one of these or any other system using multiple input methods by user interface 205. For example, a user can interact with more traditional computer human interface means, such as keyboards, mice, trackpads, and the like, as well as newer interfaces such as visual touch interfaces. In addition, some interfaces that can be used include gaming controllers, such as the “Razer Hydra” motion sensing controller by Razer Inc., with sensors 230 to detect up to the six degrees of movement above. The user interface 205 can include non-touch motion tracking interfaces that use camera technology or infra-red sensors to track objects and motion. The user interface 205 can include wearable gloves with integrated or mounted sensors that can detect motion and position of hands, such as “CyberTouch” gloves by CyberGlove Systems LLC.

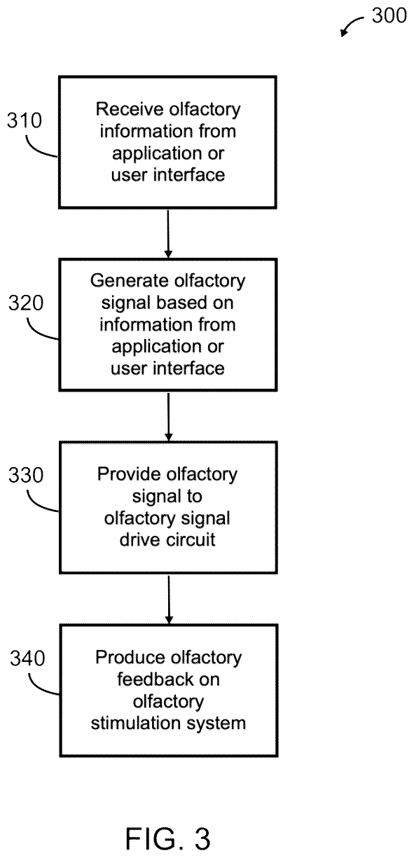

FIG. 3 is a simplified flow diagram illustrating an olfactory playback process 300 in accordance with a version of using the smell simulation system 100 of the invention. In one version, the functionality of the flow diagram of FIG. 3 is implemented by software stored in memory or other computer readable or tangible medium, and executed by the processor or controller 210. In another version, the functionality may be performed by hardware (e.g., through the use of an application specific integrated circuit (“ASIC”), a programmable gate array (“PGA”), a field programmable gate array (“FPGA”), etc.), or any combination of hardware and software, and may be internal or external to the processor or controller 210 and/or the smell simulation system 100.

At 310, olfactory information is received from an application or user interface, such as user interface 205. In the case where olfactory information is received from an application, the application can generate the olfactory information based on a user's interaction with the virtual environment 115. For example, a user can interact with an application via user interface 205. The application can interpret the user's interactions by user interface 205 and provide a corresponding reaction on a display or the like. In addition, the application can provide olfactory information based on the user's interactions and application reaction. In the case where olfactory information is received from a user interface, such as user interface 205, olfactory information can be received directly from the user interface 205. Such information may include information on the position of the user in the virtual environment 115 and/or the orientation of a user or the user's hands and perceived actions taken by the user. The olfactory information can be received by wire or wirelessly or by any other manner of communication. When the olfactory information is received wirelessly, such wireless technologies used for wireless reception can include any known types of wireless technology including those based on radio frequencies, magnetic fields, and visible and invisible electromagnetic frequencies.

At 320, the olfactory information can be processed to generate an olfactory signal indicative of the olfactory simulation to the provided to the user. For example, if the olfactory information from an application informs that a virtual hand contacted and lifted an apple, the olfactory information can be processed to generate an olfactory signal based on that information. In this situation, an olfactory signal can be a signal related to the smell of an apple.

At 330, the olfactory signal can be provided to the olfactory signal drive circuit 220. The olfactory signal drive circuit 220 can optionally translate the olfactory signal into a signal recognizable to the olfactory stimulation system 105. The olfactory signal can be provided by wire or wirelessly to the olfactory signal drive circuit 220. In one version, odor stimulation technical details can be saved in firmware.

At 340, the olfactory feedback is produced on the olfactory stimulation system 105 and is delivered to the user in proximity to the olfactory stimulation delivery device 120 by the one or more olfactory stimulators 130. The olfactory stimulation delivery device 105 can receive the olfactory drive signal by wire or wirelessly, using any known wireless technology such as those previously discussed.

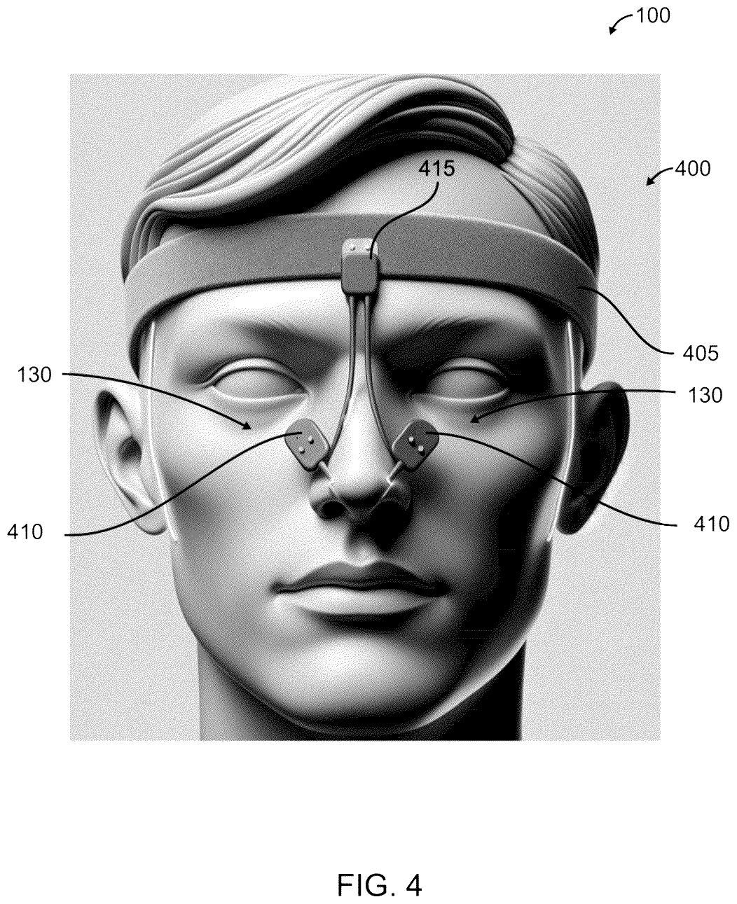

FIG. 4 shows a version of an olfactory stimulation system 105 that may be used with the smell simulation system 100 of the invention. In this version, the olfactory stimulation delivery device 120 is in the form of a head set 400 that includes a body 405 that is connectable to a head of the user or wearable on or near the head of the user. For example, in the particular version shown, the body 405 is in the form of a headband 410 or the like that fits on the head and/or forehead of a user. Connected to the body 405 of the head set 400 is the one or more olfactory stimulators 130. In the version of FIG. 4, the one or more olfactory stimulators 130 comprise one or more electrodes in the form of non-invasive electrical stimulus emitters 415 that are positionable to deliver an electrical stimulus to, near, or through an external surface of the user. The non-invasive electrical stimulus emitters 415 are designed to be able to deliver a desired electrical stimulus to at a desired location on the user. By careful selection of the stimulus and location, the olfactory region of the user can be stimulated in a manner that produces a desired smell simulation for the user. This smell simulation can be achieved without the use of chemical scents and without the use of invasive stimulation. A stimulation device controller 420 is also positioned on or near the head set 400. The stimulation device controller 420 receives the olfactory signal delivered to the olfactory stimulation system 105 by the smell simulation system 100 and/or the processor 210 and controls the electrical stimulation that is applied through the non-invasive electrical stimulus emitters 415. The stimulation device controller 415 is capable of delivering current to the user though the non-invasive electrical stimulus emitters 415. In one version, the non-invasive electrical stimulus emitters 410 are adapted to deliver electromagnetic pulses to the user. The electromagnetic pulses can be delivered to the olfactory bulb of the user and cause the brain of the user to perceive the detection of a particular smell.

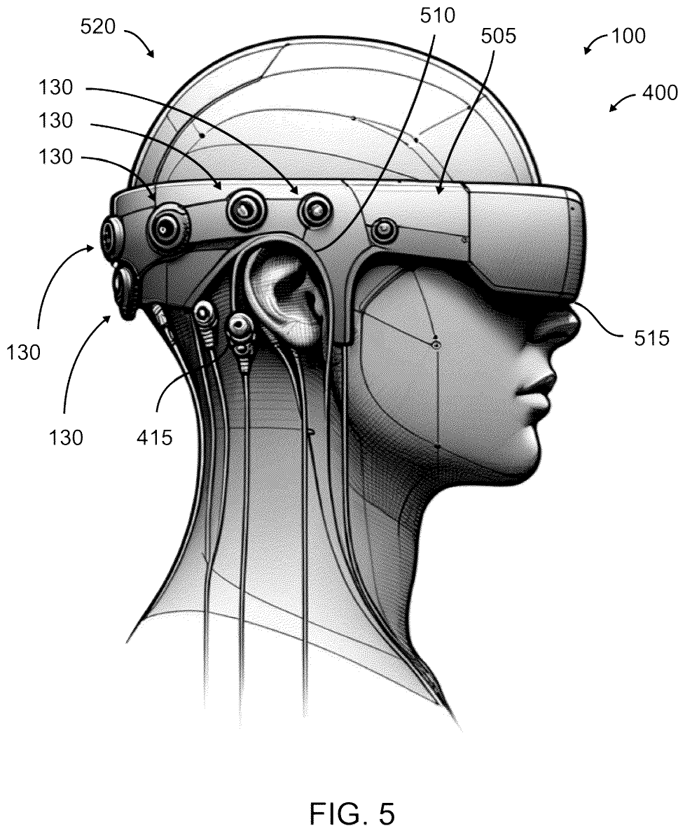

FIG. 5 shows another version of an olfactory stimulation system 105 that may be used with the smell simulation system 100 of the invention. In this version, the olfactory stimulation delivery device 120 is in the form of a head set 400 that includes a body 405 in the form of glasses or goggles 505 or the like that include an car piece 510 that can be placed atop the car of the user and/or a nose piece 515 that can rest on the bridge of the nose of the user in the manner of conventional glasses or goggles. In another version, the head set 400 can have a body 405 in the form of a virtual reality head set or the like. Various headset designs are disclosed in United States Provisional Patent Application 63/623,029 filed on Jan. 19, 2024, and each of those designs are incorporated herein by reference. In addition, each of the designs illustrate schematically possible locations for placement of the one or more olfactory stimulators 130, such as non-invasive electrical stimulus emitters 410. The design of the olfactory stimulation delivery device 120 and the olfactory stimulators 130 can be any of the shown head sets 400 and/or any combination of the shown locations and any combination of the locations along with the different style head sets 400. For each head set design, a person in the art will understand how to include a structure to position the one or more emitters 410 at a particular site. By locations it is meant any area indicated by a circle, point, line, or other clear indicator of a position on a user.

The placement of the olfactory stimulators 130, such as the non-invasive electrical stimulus emitters 410, in the present olfactory stimulation system 105 can be any position or combination of positions that will produce a desired olfactory sensation. The amount or magnitude of the electrical stimulus to be provided to simulate a smell can be determined based on the chosen site of the olfactory stimulators 130, and vice versa. While one or two emitters carefully placed can provide a desired olfactory sensation, with additional emitters and locations selected, the quality of the sensation can be increased and/or the number of possible sensations can be increased. For example, as shown in FIG. 5, the olfactory stimulators 130 can be a plurality of olfactory stimulators 130 placed in a stimulator array 520 where each olfactory stimulator 130 is positioned at a different location on the user and thus produces a different olfactory sensation by the user. Particularly useful locations or combinations of locations for placement of the one or more olfactory stimulators 130, such as one or more non-invasive electrical stimulation emitters 410, include either side of the bridge of the nose, the posterior side of the scalp, the center of the lower forehead, the neck posterior, and any other location that produces an olfactory response. These locations can be stimulated alone or in combination. In particular, combinations of locations that can be stimulated can be any one or more of the combinations of locations disclosed in US 2023/0158292 which is incorporated herein by reference in its entirety. The olfactory sensations produced by each sensor, each combination of sensors, and/or the strength or type of stimulation applied by the sensor or sensors can be empirically determined, and the combination of factors that produces a particular sensation can be saved and cataloged in the olfactory effects module 225 so that when that particular sensation is to be experienced by the user, the olfactory stimulation system 105 can be caused to produce the desires olfactory sensation in the user.

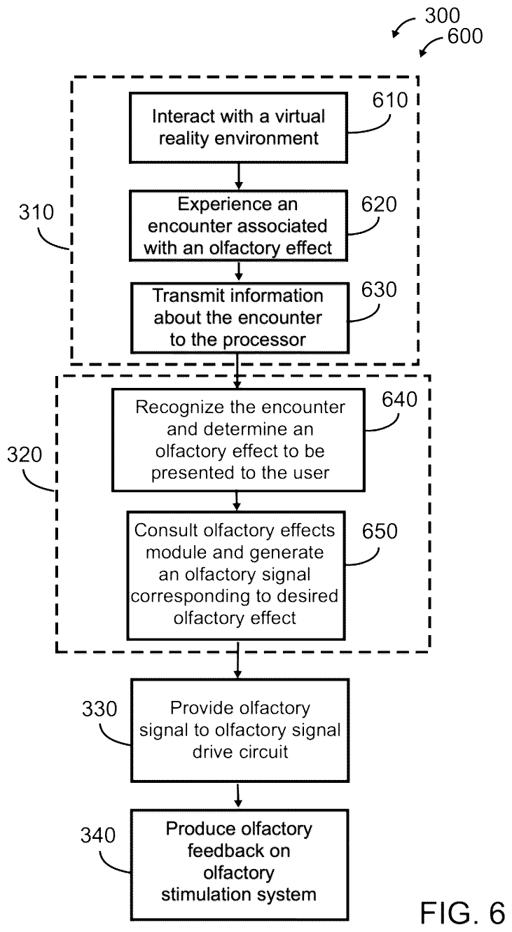

FIG. 6 is a flow diagram illustrating a particular version of an olfactory playback process 300 in accordance with a version of using the smell simulation system 100 of the invention. The version of FIG. 6 is a virtual reality interaction olfactory playback process 600. In this version, a user interacts 610 via the user interface 205 and/or sensors 230 with a virtual reality environment being run or executed on an application. When the interaction between the user and the virtual reality environment results in an encounter 620 that is associated with an olfactory effect, such as a smell, information about the olfactory effect is transmitted 630 to the processor 210. The processor 210 then recognizes 640 the encounter and determines an an olfactory effect associated with the encounter. For example, the processor 210 can recognize an encounter with an orange and can determine an olfactory effect associated with that encounter to be the scent of an orange. The processor 210 then consults 650 the olfactory effects module 225 to look up, determine, or calculate the signal or combination of signals that will produce the desired olfactory simulated effect, such as a signal or comination of signals that will produce an olfactory simulated orange scent for the user.

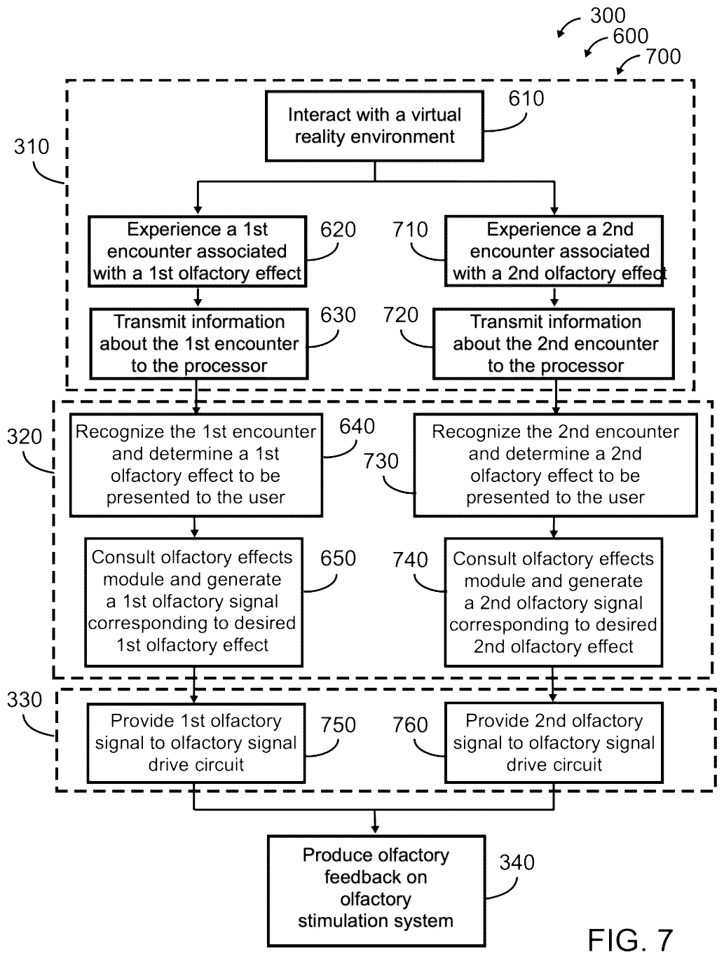

FIG. 7 shows a version of an olfactory playback process 300 similar to the version of FIG. 6 but with the process being a multi-effect virtual reality olfactory playback process 700 with multiple encounters occurring within the user's interaction with the virtual reality environment 610 and producing multiple olfactory effects. In this version, a second olfactory effect is encountered 710 during or after the first olfactory effect is encountered 620. Information about the second olfactory effect is transmitted 720 to the processor 210. The processor 210 then recognizes 730 the second encounter and determines a second olfactory effect associated with the second encounter. For example, the processor 210 can recognize a first olfactory effect of a user interacting with an orange and can then determine that a first olfactory effect of an orange scent is to be presented to the user, and the processor 210 can recognize a second olfactory effect of a user interacting with an apple and can then determine that a second olfactory effect of an apple scent is to be presented to the user. The processor 210 then consults 740 the olfactory effects module 225 to look up, determine, or calculate the signal or combination of signals that will produce the second desired olfactory effect. The step 330 of providing 330 an olfactory signal to the olfactory signal drive circuit 220 comprises providing a first olfactory signal 750 and a second olfactory signal 760 to the olfactory drive circuit 220 so that both signals can be used to produce 340 olfactory feedback. The first and second signals can be applied at the same time, in series, or the signals can be rotated. Also, when it is detected that the user is no longer encountering one or the other olfactory effects, the signal associated with that effect can be stopped.

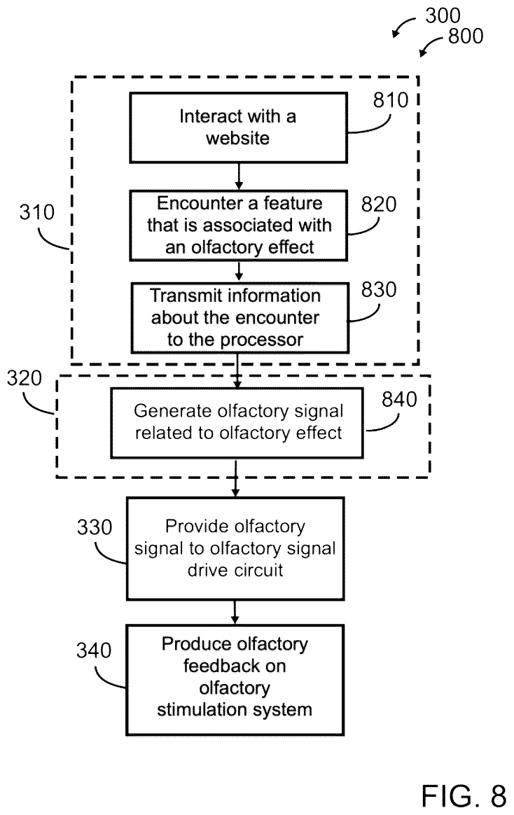

FIG. 8 is a flow diagram illustrating another particular version of an olfactory playback process 300 in accordance with a version of using the smell simulation system 100 of the invention. The version of FIG. 8 is a website interaction olfactory playback process 800. In this version, a user interacts 810 with a website and encounters 820 a feature on the website associated with an olfactory effect. For example, a user may scroll over, click on, or otherwise interact with an image that has a smell associated with the image. Information about the encounter is transmitted 830 to the processer 210, and the processor 210 generates 840 an olfactory signal related to the olfactory effect so that an olfactory signal can be provided to the user to simulate the effect. In one version, the there is a single olfactory signal that is to be applied with the single signal relating to the olfactory effect. When the processor detects the encounter, the single signal is provided to the olfactory stimulation delivery device 120. In another version, the processor 210 determines the olfactory effect to be provided to the user in similar manner to the version of FIG. 6.

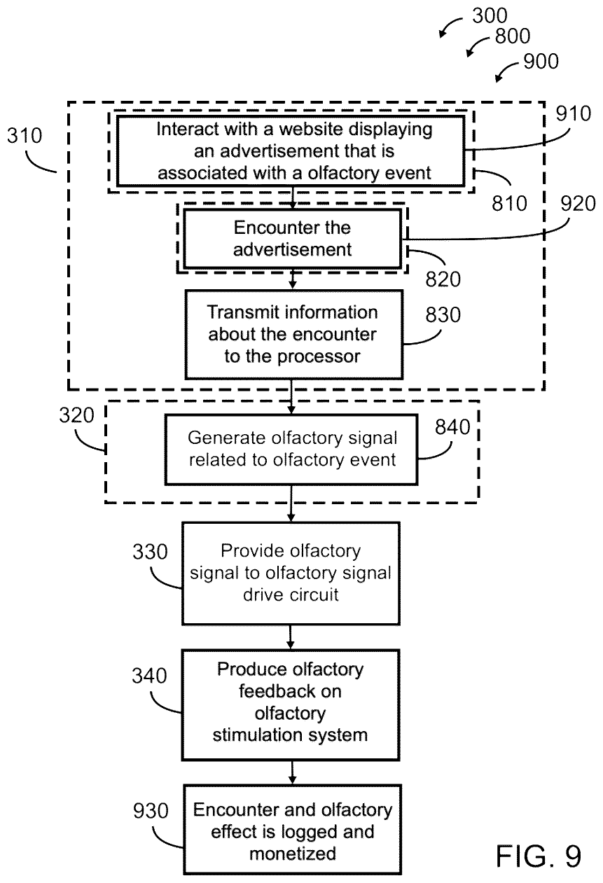

FIG. 9 is a flow diagram illustrating another particular version of an olfactory playback process 300 in accordance with a version of using the smell simulation system 100 of the invention. The version of FIG. 9 is a website interaction olfactory playback process 800 as in FIG. 8, but the version of FIG. 9 is an advertisement-based process 900. In this version, the feature on the website that is associated with an olfactory effect is an advertisement 910 on the website. When a user encounters 920 the advertisement, as discussed above, an olfactory effect related to the advertisement is provided to the user. The encounter and delivery of the olfactory effect can be logged 930 and monetized, such as by the advertiser paying the website host a fee for each such encounter similar to conventional “pay-per-click” advertising systems but for a more immersive and multisensory experience.

With the version of FIG. 9, the olfactory effect can be provided to the user by any suitable device. For example, the effect can be delivered via an olfactory stimulation delivery device 120 as disclosed herein or via a known delivery device, such as the one disclosed in U.S. Pat. No. 9,925,549 which is incorporated herein by reference. The system can be integrated with virtual environments, such as virtual reality applications, augmented reality overlays, or interactive simulations. Advertisements can appear contextually within these environments, such as a coffee aroma in a virtual café. Sensors and algorithms can monitor user behavior to determine if the user encounters the scent. Interaction may include proximity-based triggers, physiological responses (e.g., sniff detection), or explicit actions indicating scent perception. Interaction data can be transmitted to a cloud platform that processes and verifies user engagement. The data can be linked to an advertiser's account for billing purposes. A cost-per-smell (CPS) model charges advertisers based on verified scent perceptions. Advertisers receive detailed analytics, including metrics such as scent impressions, user engagement rates, and demographic insights. In use, a virtual environment hosts olfactory-enabled advertisements embedded within VR/AR experiences. When a user navigates a specific area or interacts with an object, the olfactory stimulation device releases a targeted scent. The user perceives the scent, which may complement the visual or auditory aspects of the advertisement. Sensors within the olfactory device detect and log the user's interaction, such as proximity, head movement, or explicit acknowledgment of the scent. Interaction data is sent to a cloud-based server, where it is analyzed for validity. Verification ensures that the scent was successfully delivered and perceived by the user. The system charges the advertiser based on the verified interactions. Billing occurs in real-time or at scheduled intervals, depending on the advertiser's preferences. Advertisers access a dashboard with detailed analytics, such as total impressions and verified interactions, scent-specific performance metrics, user engagement insights, including time spent in proximity to the ad. The system architecture can include an olfactory stimulation device, a virtual environment, user sensors, cloud server, advertiser dashboard. Applications and use cases include E-Commerce, such as perfume sampling in virtual stores; food and beverage, such as promoting coffee or snacks with matching aromas; gaming, such as immersive experiences with environmental scents; tourism, such as simulating destination environments, such as beach scents for travel ads; and the like.

Although the present invention has been described in considerable detail with regard to certain preferred versions thereof, other versions are possible, and alterations, permutations and equivalents of the versions shown will become apparent to those skilled in the art upon a reading of the specification and study of the drawings. For example, the cooperating components may be reversed or provided in additional or fewer number, and all directional limitations, such as up and down and the like, can be switched, reversed, or changed as long as doing so is not prohibited by the language herein with regard to a particular version of the invention. Like numerals represent like parts from figure to figure. When the same reference number has been used in multiple figures, the discussion associated with that reference number in one figure is intended to be applicable to the additional figure(s) in which it is used, so long as doing so is not prohibited by explicit language with reference to one of the figures. Also, the various features of the versions herein can be combined in various ways to provide additional versions of the present invention. Furthermore, certain terminology has been used for the purposes of descriptive clarity, and not to limit the present invention. Throughout this specification and any claims appended hereto, unless the context makes it clear otherwise, the term “comprise” and its variations such as “comprises” and “comprising” should be understood to imply the inclusion of a stated element, limitation, or step but not the exclusion of any other elements, limitations, or steps. Throughout this specification and any claims appended hereto, unless the context makes it clear otherwise, the term “consisting of” and “consisting essentially of” should be understood to imply the inclusion of a stated element, limitation, or step and the exclusion of any other elements, limitations, or steps or the exclusion of any other essential elements, limitations, or steps, respectively. Throughout the specification, any discussion of a combination of elements, limitations, or steps should be understood to include (i) each element, limitation, or step of the combination alone, (ii) each element, limitation, or step of the combination with any one or more other element, limitation, or step of the combination, (iii) an inclusion of additional elements, limitations, or steps (i.e. the combination may comprise one or more additional elements, limitations, or steps), and/or (iv) an exclusion of additional elements, limitations, or steps or an exclusion of essential additional elements, limitations, or steps (i.e. the combination may consist of or consist essentially of the disclosed combination or parts of the combination). All numerical values, unless otherwise made clear in the disclosure or prosecution, include either the exact value or approximations in the vicinity of the stated numerical values, such as for example about +/−ten percent or as would be recognized by a person or ordinary skill in the art in the disclosed context. The same is true for the use of the terms such as about, substantially, and the like. Also, for any numerical ranges given, unless otherwise made clear in the disclosure, during prosecution, or by being explicitly set forth in a claim, the ranges include either the exact range or approximations in the vicinity of the values at one or both of the ends of the range. When multiple ranges are provided, the disclosed ranges are intended to include any combinations of ends of the ranges with one another and including zero and infinity as possible ends of the ranges. Therefore, any appended or later filed claims should not be limited to the description of the preferred versions contained herein and should include all such alterations, permutations, and equivalents as fall within the true spirit and scope of the present invention.

What is claimed is:

1. A smell simulation system comprising:
an olfactory stimulation system comprising an olfactory stimulation delivery device containing an olfactory stimulator, wherein the olfactory stimulation delivery device is adapted to position the olfactory stimulator on or in proximity to the user so that the olfactory stimulator can provide an electrical stimulus to the user that generates an olfactory effect; and
a connection system adapted to connect the olfactory stimulation system to a virtual environment,
wherein the olfactory stimulation system provides an olfactory stimulation to the user in response to an interaction with the virtual environment.

2. A system according to claim 1 wherein the olfactory stimulator is adapted and positioned to provide an electrical stimulus that is delivered to the olfactory bulb or other region in the user's brain in a manner that causes the user to perceive an olfactory sensation.

3. A system according to claim 1 wherein the olfactory stimulators comprise one or more non-invasive electrical stimulus emitters.

4. A system according to claim 1 wherein the olfactory stimulation delivery device comprises a head set.

5. A system according to claim 1 wherein the olfactory effect is a simulated smell or a perceived smell sensation.

6. A system according to claim 1 wherein the olfactory stimulation system can generate a first electrical stimulation that is in response to a first interaction within the virtual environment and can generate a second electrical stimulation that is different than the first electrical stimulation and that is in response to a second interaction within the virtual environment.

7. A system according to claim 1 wherein the olfactory stimulation delivery device is designed to position one or more olfactory stimulator on one or more of either side of the bridge of the nose, the posterior side of the scalp, the center of the lower forehead, and the neck posterior.

8. A system according to claim 1 wherein the olfactory stimulation delivery device comprises a plurality of olfactory stimulators.

9. A system according to claim 1 wherein the connection system comprises an olfactory signal drive circuit that receives a signal from a processor associated with a virtual environment and uses the signal to drive the olfactory stimulation delivery device.

10. A smell simulation process comprising:
providing a virtual environment for a user; and
providing an electrical stimulation to the user in response to the user's interaction within the virtual environment,
wherein the electrical stimulation is designed to produce a simulated olfactory effect related to the virtual interaction.

11. A process according to claim 10 wherein the simulated olfactory effect is a simulated smell or a perceived smell sensation.

12. A process according to claim 10 wherein the virtual environment is provided by a virtual reality system, extended reality system, augmented reality system, mixed reality system, or gaming system.

13. A process according to claim 12 wherein the user's interaction comprises experiencing a virtual encounter associated with an olfactory effect, and wherein the electrical stimulation is designed to simulate the olfactory effect.

14. A process according to claim 12 wherein the user's interaction comprises experiencing a first virtual encounter associated with a first olfactory effect, wherein the electrical stimulation is designed to simulate the first olfactory effect, wherein the user's interaction comprises experiencing a second virtual encounter associated with a second olfactory effect different than the first olfactory effect, and wherein the electrical stimulation is designed to simulate the second olfactory effect.

15. A process according to claim 10 wherein the virtual environment is a website.

16. A process according to claim 15 wherein the user's interaction comprises encountering a feature on the website associated with an olfactory effect, and wherein the electrical stimulation is designed to simulate the olfactory effect.

17. A process according to claim 16 wherein the feature is an advertisement and wherein the provision of the electrical stimulation is logged and monetized.

18. A smell simulation process comprising:
providing a virtual environment for a user; and
providing olfactory stimulation to the user in response to the user's interaction with an advertisement within the virtual environment,
wherein the olfactory stimulation is designed to produce a simulated olfactory effect related to the advertisement.

19. A process according to claim 18 within the olfactory stimulation is an electrical stimulation, wherein the electrical stimulation is designed to produce a simulated olfactory effect related to the advertisement.

20. A process according to claim 18 further comprising logging and monetizing the olfactory stimulation.
